(19) United States
(12) Patent Application Publication
(10) Pub. No.: US 2026/0274551 A1
(43) Pub. Date: Sep. 17, 2026

(54) RHEOLOGICAL FILLING SYRINGE

(71) Applicant: Clear IP Corporation, Stafford, TX (US)

(72) Inventor: Jeff WU, Stafford, TX (US)

(73) Assignee: Clear IP Corporation, Stafford, TX (US)

(21) Appl. No.: 19/471,219

(22) PCT Filed: Mar. 28, 2024
(86) PCT No.: PCT/US2024/022062

§ 371 Date: Sep. 30, 2025

Related U.S. Application Data
(60) Provisional application No. 63/455,803, filed on Mar. 30, 2023.

(51) Int. Cl. B65D 83/76

(52) U.S. Cl. CPC B65D 83/76

(57) ABSTRACT

A rheological filling syringe is provided. The rheological filling syringe includes a body and a tip portion coupled with the body. The tip portion forms a tip such that the tip portion is operable to permit rheological material to be expelled from the body through the tip. The tip portion includes a taper portion that tapers towards the tip.

### CROSS-REFERENCE TO RELATED APPLICATION(S)

This application is a U.S. National Phase application of PCT/US2024/022062 filed Mar. 28, 2024, which claims the benefit of U.S. Provisional Patent Application No. 63/455,803, filed in the U.S. Patent and Trademark Office on Mar. 30, 2023, which is incorporated herein by reference in its entirety for all purposes.

### FIELD

The present disclosure relates generally to a rheological filling syringe. In at least one example, the present disclosure relates to a rheological filling system that includes a rheological filling syringe.

### BACKGROUND

Semi-solid viscoelastic materials in a variety of industries have complex flow and rheological properties. Flow behavior as defined by Newtonian, shear thickening or thinning, Bingham and other mathematical models change dramatically in the presence of solids, pressure, and/or temperature variables.

### BRIEF DESCRIPTION OF THE DRAWINGS

Further features of the present disclosure will become apparent to those skilled in the art to which the present disclosure relates from reading the following specification with reference to the accompanying drawings, in which:

FIG. 1 is a perspective view of a rheological filling syringe;

FIG. 2 is a side view of the rheological filling syringe in a dispel configuration;

FIG. 3 is a side view of the rheological filling syringe in a withdraw configuration;

FIG. 4 is a cross-sectional view of the rheological filling syringe of FIG. 3 taken along line A-A;

FIG. 5 is an enlarged view of circle B as shown in FIG. 4; and

FIG. 6 is a rheological filling system with the rheological filling syringe.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, substantially cylindrical means that the object resembles a cylinder, but can have one or more deviations from a true cylinder. The term “comprising” means “including, but not necessarily limited to”; it specifically indicates open-ended inclusion or membership in a so-described combination, group, series and the like.

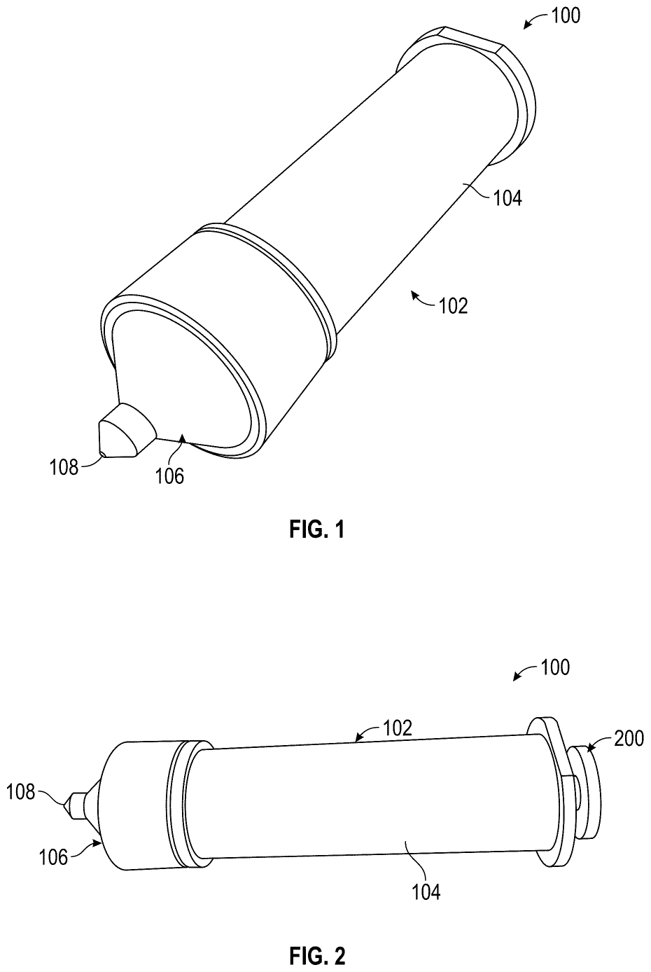

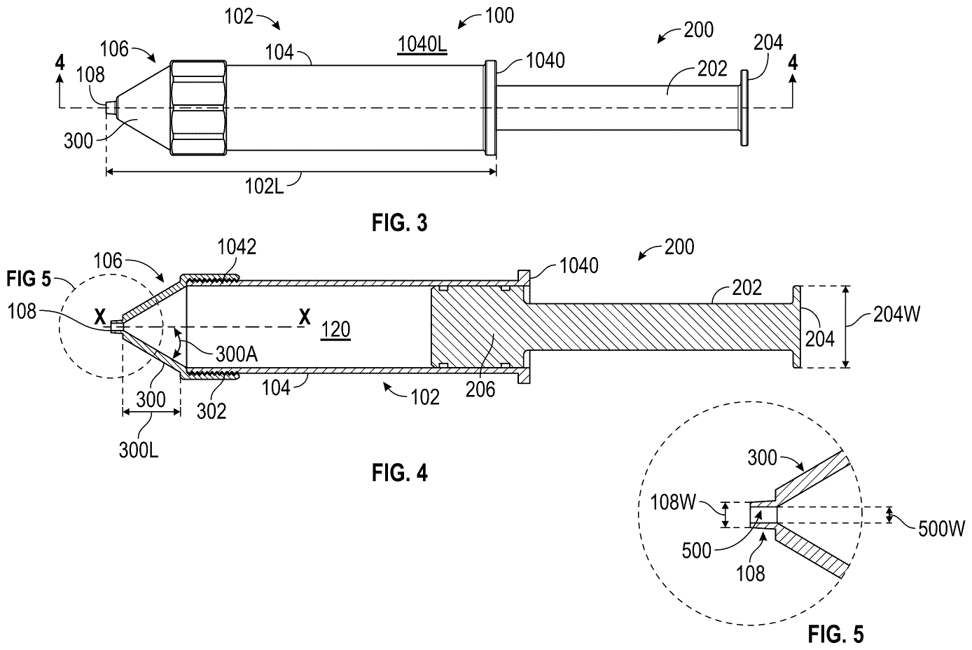

FIGS. 1-5 illustrate a rheological filling syringe 100. FIG. 1 illustrates a perspective view of the rheological filling syringe 100, FIG. 2 illustrates a side view of the rheological filling syringe 100, FIG. 3 illustrates a side view of the rheological filling syringe 100 in a withdraw configuration, FIG. 4 illustrates a cross-sectional view of the rheological filling syringe 100 of FIG. 3 taken along line A-A, and FIG. 5 illustrates an enlarged view of circle B as shown in FIG. 4.

The rheological filling syringe 100 includes receiving portion 102 that can include a body 104 and a tip portion 106. The receiving portion 102 can receive and expel material into a desired receptacle. In at least one example, the receiving portion 102 can have a length 102L between about 120 millimeters and about 250 millimeters. In some examples, the receiving portion 102 can have a length 102L between about 140 millimeters and about 220 millimeters. In some examples, the receiving portion 102 can have a length 102L between about 160 millimeters and about 190 millimeters. In some examples, the receiving portion can have a length 102L of about 179 millimeters.

In at least one example, the rheological filling syringe 100 is operable to receive material in the body 104 and expel material out of the tip portion 106. In at least one example, the material can include rheological material. Rheological material can include a material that can change its physical state in reaction to a stimulus, for example solids, pressure, and/or temperature. In at least one example, the material can include hash. In at least one example, the body 104 can include one or more walls 104 that form an annulus 120 operable to receive the material. The tip portion 106 can include a tip 108 through which the material can be expelled out of. In at least one example, the tip 108 can have a width 108W between about 3 millimeters and about 8 millimeters. In some examples, the tip 108 can have a width 108W between about 4 millimeters and about 5 millimeters. In some examples, the tip 108 can have a width 108W of about 4.84 millimeters.

The body 104 can include a rear end 1040 that is opposite a front end 1042. The front end 1042 can be operable to be coupled with the tip portion 106. In at least one example, the tip portion 106 can be removably coupled with the front end 1042 of the body 104. For example, as shown in FIG. 4, the tip portion 106 and the body 104 can each include threads 302 that are operable to removably couple the tip portion 106 with the body 104. As the tip portion 106 is removable and separable from the body 104, the tip portion 106 and/or the body 104 can be cleaned. In at least one example, the tip portion 106 can be separated from the body 104 so that the rheological material can be deposited into the annulus 120 of the body 104. In at least one example, as shown in FIG. 5, the tip 108 can form a passageway 500 that is in communication with the annulus 120 of the body 104. Accordingly, the material that is received in the annulus 120 of the body 104 can be expelled from the rheological filling syringe 100 via the passageway 500 of the tip 108. In at least one example, the passageway 500 can have a width 500W between about 2 millimeters and about 5 millimeters. In some examples, the passageway 500 can have a width 500W between about 3 millimeters and about 4 millimeters. In some examples, the passageway 500 can have a width 500W of about 3.10 millimeters.

A plunger 200, as shown in FIGS. 2-4, is operable to transition from a withdraw configuration (as shown in FIGS. 3 and 4) to a dispel configuration (as shown in FIGS. 1 and 2) to cause the material to be expelled from the body 104 through the tip 108. In at least one example, the plunger 200 can include a seal portion 206 received in the annulus 120 of the body 120, a shaft 202 coupled to the seal portion 206 that can extend from a rear end 1040 of the body 120, and a base 204. In at least one example, the base 204 can have a width 204W between about 25 millimeters and about 45 millimeters. In some examples, the base 204 can have a width 204W between about 30 millimeters and about 40 millimeters. In some examples, the base 204 can have a width 204W of about 35 millimeters. The seal portion 206 is operable to create a seal against the walls 104 of the body 104 so that any material such as a fluid and/or solid (e.g., rheological and/or rheological oil) does not pass across the seal portion 206. Accordingly, when the plunger 200 is pushed to transition from the withdraw configuration to the dispel configuration, the seal portion 206 pushes against the material such that the material is pushed towards the tip portion 106 and out of the tip 108.

When the plunger 200 is transitioning to the dispel configuration, the plunger 200 imparts a pressure on the rheological material disposed in the body 104 and the tip portion 106. The pressure can be substantial to cause the rheological material to separate into oil and solid when the rheological material is exposed to the pressure. For example, the pressure can be between about 400 pounds and about 600 pounds. In some examples, the pressure can be about 500 pounds. When the material is separated into oil and solid, then oil may be expelled from the tip 108 while solid material may remain in the tip portion 106. Accordingly, the concentration of the material being deposited from the rheological filling syringe 100 may be inconsistent.

To reduce the pressure imparted on the rheological material in the tip portion 106, the tip portion 106 can include a taper portion 300 that tapers towards the tip 108. The taper portion 300 has a narrowing width and/or diameter as the taper portion 300 approaches the tip 108. The taper portion 300 can taper towards the tip 108 at an angle 300A formed between a tapered wall 301 and a central axis X-X. In at least one example, the angle 300A can be between about 15 degrees and about 60 degrees. In some examples, the angle 300A can be between about 25 degrees and about 45 degrees. In some examples, the angle 300A can be between 29 degrees and about 31 degrees. In some examples, the angle 300A can be about 30 degrees. In at least one example, the taper portion 300 can have a length 300L between about 15 millimeters and about 35 millimeters. In some examples, the taper portion 300 can have a length 300L between about 20 millimeters and about 30 millimeters. In some examples, the taper portion 300 can have a length 300L of about 25 millimeters. The taper portion 300 can be operable to reduce an amount of the rheological separating into oil and solid when the rheological material is exposed to the pressure from the plunger 200. Accordingly, the concentration of the rheological material being expelled from the rheological filling syringe 100 is more consistent and calculable.

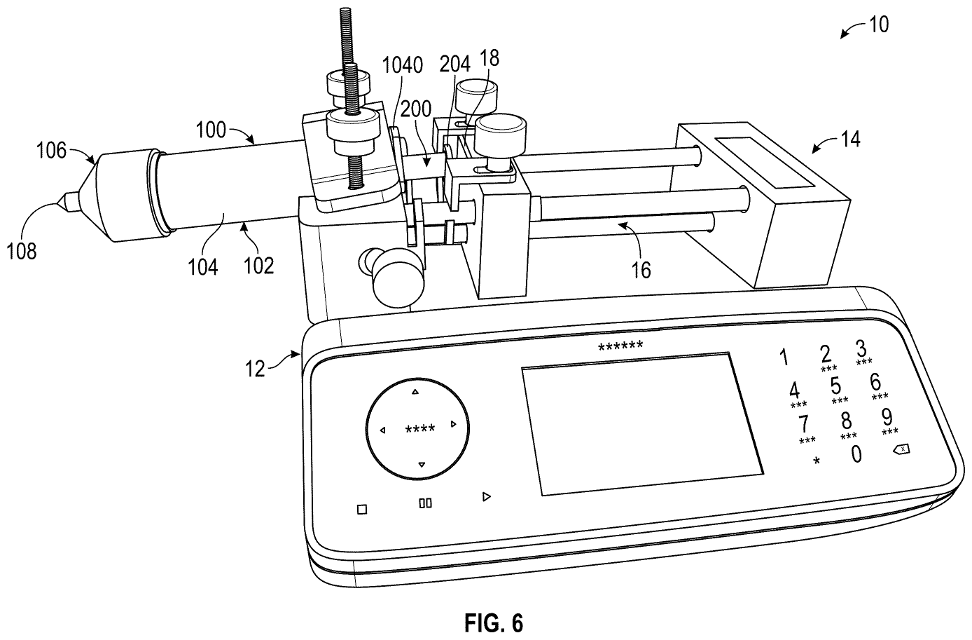

FIG. 6 illustrates a rheological filling system 10 operable to hold the rheological filling syringe 100 can cause the rheological filling syringe 100 to transition from the withdraw configuration to the dispel configuration to expel the material from the tip 108. The rheological filling system 10 can impart a force on the plunger 200 of the rheological filling syringe 100 to cause the plunger 200 to transition to the dispel configuration and push the material out of the tip 108 of the rheological filling syringe 100.

The rheological filling system 10 can include a holding component 12 operable to receive the rheological filling syringe 100. The holding component 12 can maintain the position of the receiving portion 102 of the rheological filling syringe 100 while the plunger 200 transitions to the dispel configuration. In at least one example, the holding component 12 can include a clamp operable to receive and hold the position of the rheological filling syringe 100.

A pushing component 14 can be operable to transition the rheological filling syringe 100 to cause a predetermined amount of material to be expelled from the body 104 through the tip 106. The pushing component 14 can include a plurality of pistons 16 that are operable to extend and push the plunger 200 of the rheological filling syringe 100 to transition to the dispel configuration. In at least one example, the pistons 16 can be powered by a motor. In some examples, the pistons 16 can be hydraulicly powered. Other mechanisms to power the pistons 16 can be utilized without deviating from the scope of the disclosure. In some examples, the pistons 16 can be coupled to and/or abut a receiving component 18. The receiving component 18 can be operable to receive at least a portion of the plunger 200 (e.g., the shaft 202 and/or the base 204). Accordingly, when the pistons 16 push the receiving component 18, the receiving component 18 moves and causes the plunger 200 to transition to the dispel configuration.

In at least one example, the rheological filling system 10 can be set to cause the rheological filling syringe 100 to expel a predetermined amount of material. Accordingly, the amount of material being expelled from the rheological filling syringe 100 is controlled and consistent. With the addition of the taper portion 300 of the tip portion 106, the concentration and the amount of the material being expelled from the rheological filling syringe 100 can be consistently and predictably controlled.

Although a variety of information was used to explain aspects within the scope of the appended claims, no limitation of the claims should be implied based on particular features or arrangements, as one of ordinary skill would be able to derive a wide variety of implementations. Further and although some subject matter may have been described in language specific to structural features and/or method steps, it is to be understood that the subject matter defined in the appended claims is not necessarily limited to these described features or acts. Such functionality can be distributed differently or performed in components other than those identified herein. Rather, the described features and steps are disclosed as possible components of systems and methods within the scope of the appended claims.

What is claimed is:

1. A rheological filling syringe comprising:
a body;
a tip portion coupled with the body, the tip portion forming a tip such that the tip portion is operable to permit rheological material to be expelled from the body through the tip, the tip portion including a taper portion that tapers towards the tip.

2. The rheological filling syringe of claim 1, wherein the taper portion tapers towards the tip at an angle formed between a tapered wall and a central axis.

3. The rheological filling syringe of claim 2, wherein the angle is between about 15 degrees and about 70 degrees.

4. The rheological filling syringe of claim 2, wherein the angle is between about 25 degrees and about 45 degrees.

5. The rheological filling syringe of claim 2, wherein the angle is between about 29 degrees and about 31 degrees.

6. The rheological filling syringe of claim 1, further comprising a plunger operable to transition from a withdraw configuration to an dispel configuration to cause the rheological material to be expelled from the body through the tip.

7. The rheological filling syringe of claim 6, wherein the plunger transitioning to the dispel configuration imparts a pressure on the rheological material disposed in the body and the tip portion.

8. The rheological filling syringe of claim 7, wherein the pressure is between about 400 pounds and about 600 pounds.

9. The rheological filling syringe of claim 7, wherein the taper portion is operable to reduce an amount of the rheological material to separate into oil and solid when the rheological material is exposed to the pressure.

10. The rheological filling syringe of claim 7, wherein the taper portion is operable to reduce the pressure imparted on the rheological material in the tip portion.

11. A rheological filling system comprising:
a rheological filling syringe including:
a body;
a tip portion coupled with the body, the tip portion forming a tip such that the tip portion is operable to permit rheological material to be expelled from the body through the tip, the tip portion including a taper portion that tapers towards the tip;
a holding component operable to receive the rheological filling syringe; and
a pushing component operable to transition the rheological filling syringe to cause a predetermined amount of rheological material to be expelled from the body through the tip.

12. The rheological filling system of claim 11, wherein the taper portion tapers towards the tip at an angle formed between a tapered wall and a central axis.

13. The rheological filling system of claim 12, wherein the angle is between about 60 degrees and about 15 degrees.

14. The rheological filling system of claim 12, wherein the angle is between about 25 degrees and about 45 degrees.

15. The rheological filling system of claim 12, wherein the angle is between about 29 degrees and about 31 degrees.

16. The rheological filling system of claim 11, wherein the rheological filling syringe further includes a plunger, wherein the pushing component is operable to cause the plunger to transition from a withdraw configuration to an dispel configuration to cause the rheological material to be expelled from the body through the tip.

17. The rheological filling system of claim 16, wherein the plunger transitioning to the dispel configuration imparts a pressure on the rheological material disposed in the body and the tip portion.

18. The rheological filling system of claim 17, wherein the pressure is between about 400 pounds and about 600 pounds.

19. The rheological filling system of claim 17, wherein the taper portion is operable to reduce an amount of the rheological material to separate into oil and solid when the rheological material is exposed to the pressure.

20. The rheological filling system of claim 17, wherein the taper portion is operable to reduce the pressure imparted on the rheological material in the tip portion.
